(19) United States
(12) Patent Application Publication
(10) Pub. No.: US 2010/0160295 A1
(43) Pub. Date: Jun. 24, 2010

(54) TREATMENT OF RESTLESS LEG SYNDROME AND SLEEP DISORDERS

(75) Inventors: Lesley PICKFORD, Menlo Park, CA (US); Uwe Meya, South San Francisco, CA (US); Mark Moran, South San Francisco, CA (US)

Correspondence Address: MORGAN, LEWIS & BOCKIUS LLP (SF), One Market, Spear Street Tower, Suite 2800, San Francisco, CA, 94105, US

(73) Assignee: SYNOSIA THERAPEUTICS, South San Francisco, CA (US)

(21) Appl. No.: 12/614,390

(22) Filed: Nov. 6, 2009

Related U.S. Application Data
(60) Provisional application No. 61/111,955, filed on Nov. 6, 2008.

(51) Int. Cl. A61K 31/122; A61K 31/485; A61K 31/55; A61K 31/48; A61K 31/197; A61K 31/195; C07C 205/46; A61P 25/00

(52) U.S. Cl. 514/217; 514/676; 514/282; 514/288; 514/565; 514/561; 568/306

(57) ABSTRACT

Methods of preventing and/or treating at least one symptom of Restless Leg Syndrome (RLS) and its related disorders, including disorders such as periodic limb movements in sleep (PLMS) and periodic limb movement disorder (PLMD), are disclosed. The methods comprise identifying a host, afflicted with Restless Leg Syndrome (RLS) and its related disorders; and administering to the host a pharmaceutically effective amount of a 4-hydroxyphenylpyruvate dioxygenase. Also provided are methods and compositions for treating/preventing sleep disorders, and methods and compositions for improving sleep quantity and adequacy.

### CROSS-REFERENCE TO RELATED APPLICATIONS

This application claims the benefit of U.S. Provisional Pat. App. No. 61/111,955, filed Nov. 6, 2008 which is incorporated by reference in its entirety for all purposes.

### FIELD OF THE INVENTION

The present invention relates to novel methods for treating or preventing Restless Leg Syndrome (RLS), and related disorders, and for the occurrence of unwanted limb movements. In exemplary embodiments, this invention relates to the use of an inhibitor of 4-hydroxyphenylpyruvate dioxygenase (HPPD), and in exemplary embodiments to the use of 2-(2-nitro-4-trifluoromethylbenzoyl)-1,3-cyclohexanedione, for treating or preventing RLS and/or related disorders. In exemplary embodiments, the present invention also provides methods and compositions for treating or preventing sleep disorders, and improving sleep quantity and adequacy by administering an HPPD inhibitor to a subject.

### BACKGROUND OF THE INVENTION

Restless Leg Syndrome (RLS) is a sensorimotor disorder where an individual suffering from RLS has an unpleasant sensation in the legs at rest (or during inactivity), causing what is often described as an irresistible desire to move, which generally alleviates the discomfort. In addition, individuals afflicted with RLS experience crawling sensations in their legs that often occur at night and that are only relieved by moving the legs. RLS was first described in 1945, with an estimated prevalence of 5%. More recent studies have suggested prevalence rates of between 3% and 15%, although prevalence may be as high as 24% in certain patient groups. Many sufferers go undiagnosed and untreated, although the introduction of standardized criteria for diagnosis of RLS (updated in 2003) has improved this situation.

The clinical criteria for diagnosis, approved by the International Restless Leg Syndrome Study Group, include sleep disturbance, involuntary movements in sleep or wakefulness, a normal neurologic examination, a chronic clinical course, and, in some cases, a positive family history. Clinically, RLS is indicated when the following four minimal criteria for diagnosis are met: (1) desire to move the extremities, often associated with paresthesias/dysesthesias; (2) motor restlessness to reduce sensations; (3) worsening of symptoms at rest, or during inactivity, with at least temporary relief by activity, and (4) worsening of symptoms in the evening or night. The related disorders share some of these characteristics.

RLS may be subdivided into primary (idiopathic) and secondary RLS. Secondary RLS is commonly associated with metabolic disorders or conditions that result in iron-deficiency anemia, and may be treated by iron replacement therapy. Primary RLS is a heterogeneous disease with multiple potential causes, but recent studies have suggested that an underlying defect in dopaminergic function may be common to most forms of primary RLS. In particular, there is evidence for a decrease in the number or affinity of dopamine D2 receptors in the striatum of RLS patients. The underlying cause of RLS and its related disorders is not clearly known, but it has been observed that the frequency of occurrence increases with age. In most individuals with RLS, diagnostic results of complete blood cell counts and iron, ferritin, folate, and vitamin B12 levels do not indicate hematologic or chemical abnormalities compared with those who do not have RLS. An evaluation of the efficacy of certain drugs revealed that the dopaminergic, adrenergic and opiate systems play a role in the pathogenesis of RLS.

Current treatments for RLS are limited and only four FDA-approved medications are available (the dopamine agonists ropinirole, pramipexole, pergolide and carbidopa/levidopa). Treatment options commonly employed include the use of benzodiazipines (e.g., clonazepam), narcotics, dopamine agonists, clonidine, gabepentin and magnesium. However, these treatments have undesirable side effects, notable rebound (the return of symptoms as the medication wears off) and augmentation (a worsening of symptoms following initiation of therapy). Rebound is a particular problem with medications with a short plasma half-life, e.g. levidopa, and may occur in over 35% of patients. Augmentation is a more serious side effect, and is estimated to occur in 50-85% of patients taking levidopa, and 30-32% of patients using dopamine agonists. Benzodiazepines, opiates and anti-convulsants are not as uniformly effective as the dopamine agents, and have undesirable side effects including tolerance, dependency and GI disturbances Pramipexole, another popular therapeutic agent, has been reported to cause major side effects including insomnia, dizziness, constipation, asthenia and hallucinations.

Therefore, there exists a need for an effective, alternative treatment and related treatment regime options for individuals who are afflicted with RLS and/or its related disorders. More particularly, there exists a need for treatments that do not induce the unwanted effects observed in modern therapeutics for Restless Leg Syndrome (RLS) and related disorders.

### SUMMARY OF THE INVENTION

In various embodiments, the present invention provides compositions and methods for the treatment or prevention of diseases, such as restless leg syndrome (RLS), including disorders such as periodic limb movements in sleep (PLMS) and periodic limb movement disorder (PLMD), by partially or completely inhibiting 4-hydroxyphenylpyruvate dioxygenase (HPPD) in a subject with such a disease. In exemplary embodiments, the invention also provides compositions and methods for improvement of sleep quantity and adequacy, and treatment of sleep disorders by administering a therapeutically effective amount of an HPPD inhibitor to a subject.

This invention encompasses methods of treating and preventing disorders and conditions that are ameliorated by the inhibition of HPPD, which comprise administering to a patient in need of such treatment or prevention a therapeutically or prophylactically effective amount of a HPPD inhibitor. An exemplary HPPD inhibitor is 2-(2-nitro-4-trifluoromethylbenzoyl)-1,3-cyclohexanedione. In specific methods of the invention, the HPPD inhibitor is optionally administered in combination with an additional pharmacologically active compound.

This invention further encompasses pharmaceutical compositions and dosage forms which can be used, for example, in the methods disclosed herein. Exemplary pharmaceutical compositions of the invention comprise a therapeutically or prophylactically effective amount of a HPPD inhibitor and optionally an additional pharmacologically active compound.

These and other aspects of the present invention will become evident upon reference to the following detailed description. In addition, various references are set forth herein which describe in more detail certain procedures or compositions, and are therefore incorporated by reference in their entirety.

### BRIEF DESCRIPTION OF THE DRAWINGS

FIG. 1 is a graphical display of sleep parameters showing improvement in these parameters upon administration of an HPPD inhibitor

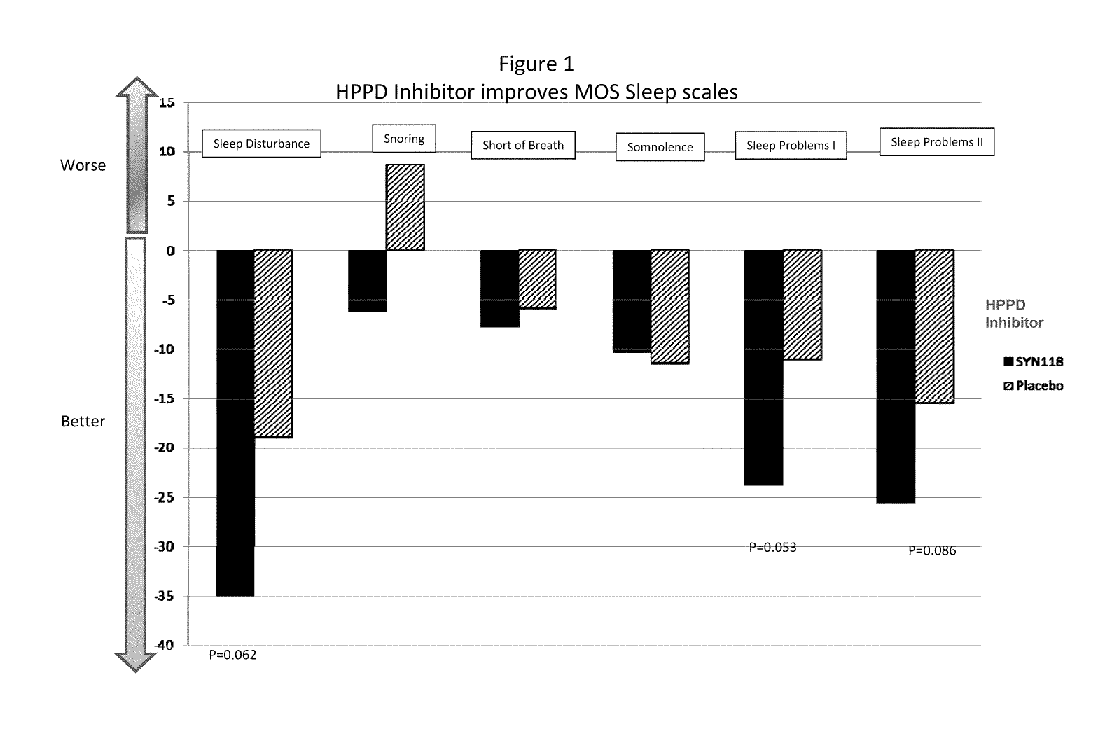

FIG. 2 is a graphical display showing improvement in sleep quantity and sleep adequacy upon administration of an HPPD inhibitor.

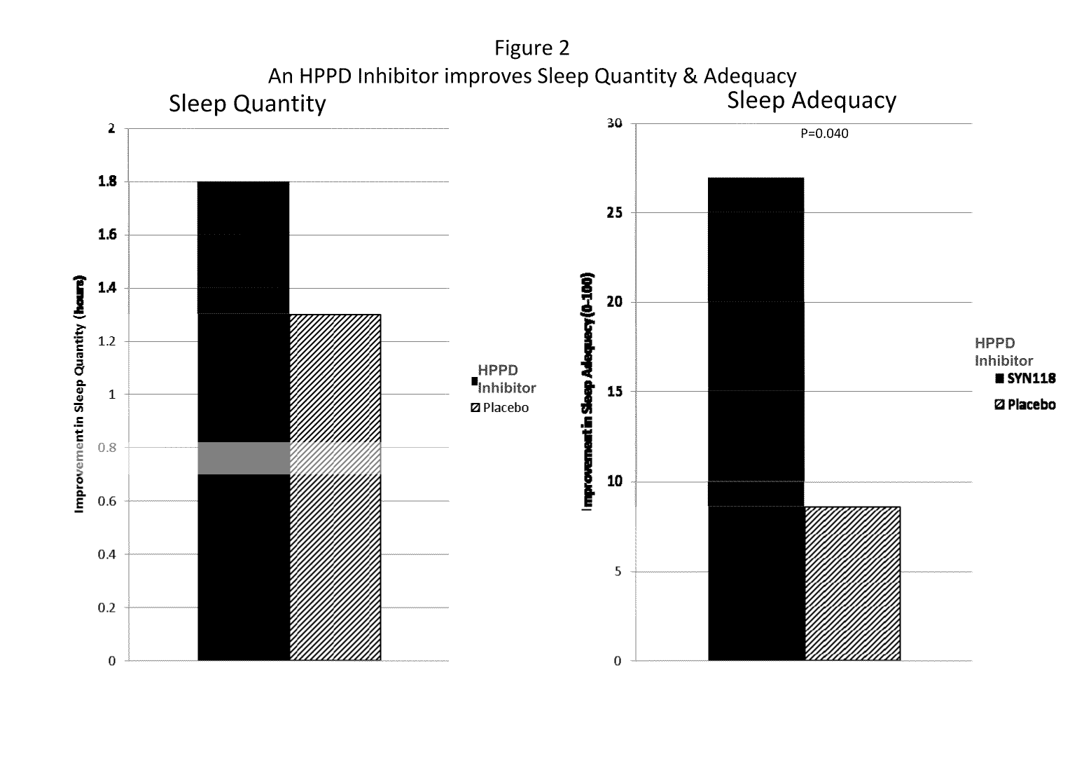

FIG. 3 is a graphical comparison of the mean plasma level of an exemplary HPPD inhibitor with plasma tyrosine at steady state following 2 mg QD vs. 2 mg Q2D dosing.

### DETAILED DESCRIPTION OF THE INVENTION

### I. Definitions

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Definition of standard chemistry terms may be found in reference works, including Carey and Sundberg (1992) “Advanced Organic Chemistry 3^{rd} Ed.” Vols. A and B, Plenum Press, New York. The practice of the present invention will employ, unless otherwise indicated, conventional methods of mass spectroscopy, protein chemistry, biochemistry, recombinant DNA techniques and pharmacology, within the skill of the art.

The term “agonist” means a molecule such as a compound (e.g., a drug, an enzyme activator or a hormone) that enhances the activity of another molecule or that bind to 4-hydroxyphenylpyruvate dioxygenase (HPPD).

The term “antagonist” means a molecule such as a compound (e.g., a drug, an enzyme inhibitor, or a hormone) that diminishes or prevents the action of another molecule or that binds to HPPD.

The term “inhibitor,” as in “enzyme inhibitor,” refers to a compound that binds to a site on an enzyme inactivating, reducing, or otherwise altering the activity of the enzyme. An exemplary enzyme inhibitor of use in the invention is an HPPD inhibitor that does not also agonize HPPD. Another exemplary enzyme inhibitor of use in the invention is an HPPD inhibitor that does not antagonize HPPD. An exemplary enzyme inhibitor of use in the invention is a highly specific and potent inhibitor of HPPD.

The terms “effective amount” or “pharmaceutically effective amount” refer to a nontoxic but sufficient amount of the agent to provide the desired biological result and, which has a clinically acceptable safety margin with respect to doses/amounts which can produce unacceptable adverse effects (e.g., toxicity) a subject (e.g., a human). That result can be reduction and/or alleviation of the signs, symptoms, or causes of a disease, or any other desired alteration of a biological system. For example, an “effective amount” for therapeutic uses is the amount of the composition comprising an inhibitor for HPPD disclosed herein required to provide a clinically significant decrease in RLS, including disorders such as periodic limb movements in sleep (PLMS) and periodic limb movement disorder (PLMD). An appropriate “effective” amount in any individual case may be determined by one of ordinary skill in the art using routine experimentation.

As used herein, the terms “treat” or “treatment” are used interchangeably and are meant to indicate a postponement of development of RLS and/or a reduction in the severity of such symptoms that will or are expected to develop. Also included is the treatment or prevention of sleep disorders or abnormality, and the improvement of sleep quantity or adequacy. The terms further include ameliorating existing symptoms of RLS, preventing additional symptoms, alleviating or preventing the symptoms of sleep disorders (e.g., sleep disturbance), ameliorating or preventing the underlying metabolic causes of symptoms of RLS and sleep disorders. Also included is a method for improving sleep quantity and/or adequacy by administering to a subject in need of a therapeutically effective amount of an HPPD inhibitor.

The phrases “sleep disorders,” “sleep abnormality,” “sleep quantity” and “sleep adequacy,” as used herein, have the meanings normally ascribed to them in the art. These parameters can be measured and quantified by art-recognized tools, e.g., the MOS Sleep Scale. Examples of “sleep disorders” or “sleep abnormalities” include primary insomnia; secondary insomnia; situational insomnia; transient insomnia; short-term insomnia; chronic insomnia; acute insomnia; prolonged latency to sleep onset; difficulty falling asleep; difficulty staying asleep; sleep maintenance problems, including without limitation, frequent awakenings, an increase in time spent awake after initially falling asleep (wake time after sleep onset, or WASO), sleep fragmentation, transient microarousals, and unrefreshing sleep; increased time awake during the sleep period; waking up too early; and reduced total sleep time.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

By “physiological pH” or a “pH in the physiologically acceptable range” is meant a pH in the range of approximately 7.2 to 8.0 inclusive, more typically in the range of approximately 7.2 to 7.6 inclusive.

As used herein, the term “subject” encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. The term does not denote a particular age or gender. When the compositions and methods of the invention are used to treat or prevent RLS or sleep disorders, or to improve sleep quantity or adequacy, exemplary subjects are not otherwise in need of treatment with an HPPD inhibitor. An exemplary subject treated with the compositions and/or methods of the invention is one who is not otherwise in need of treatment with 2-(2-nitro-4-trifluoromethylbenzoyl)-1,3-cyclohexanedione. An exemplary subject treated with the compositions/methods of the invention is one who does not have tyrosinemia (e.g., hereditary tyrosinemia). A further exemplary subject treated with the compositions/methods is one that does not otherwise have a neurodegenerative disease, which is, for example, treatment by administration of an HPPD inhibitor, e.g., Parkinson's.

The term “pharmaceutically acceptable salt” of a compound means a salt that is pharmaceutically acceptable and that possesses the desired pharmacological activity of the parent compound. Such salts, for example, include:
(1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like;
(2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine, tromethamine, N-methylglucamine, and the like. Acceptable inorganic bases include aluminum hydroxide, calcium hydroxide, potassium hydroxide, sodium carbonate, sodium hydroxide, and the like. It should be understood that a reference to a pharmaceutically acceptable salt includes the solvent addition forms or crystal forms thereof, particularly solvates or polymorphs. Solvates contain either stoichiometric or non-stoichiometric amounts of a solvent, and are often formed during the process of crystallization. Hydrates are formed when the solvent is water, or alcoholates are formed when the solvent is alcohol. Polymorphs include the different crystal packing arrangements of the same elemental composition of a compound. Polymorphs usually have different X-ray diffraction patterns, infrared spectra, melting points, density, hardness, crystal shape, optical and electrical properties, stability, and solubility. Various factors such as the recrystallization solvent, rate of crystallization, and storage temperature may cause a single crystal form to dominate.

The term “optional” or “optionally” means that the subsequently described event or circumstance may or may not occur, and that the description includes instances where the event or circumstance occurs and instances where it does not. For example, the phrase “optionally an additional pharmacologically active compound” means that the patient may or may not be given a drug other than the inhibitors of HPPD.

“Additional pharmacologically active compound” as used herein is meant any chemical material or compound suitable for administration to a mammalian, preferably human, which induces a desired local or systemic effect, such as, for example, narcotics, dopamine agonists, benzodiazipines (e.g., clonazepam), clonidine, gabepentin, magnesium, iron, and vitamins, such as vitamin B12. In general, this includes: anorexics; anti-infectives such as antibiotics and antiviral agents, including many penicillins and cephalosporins; analgesics and analgesic combinations; antiarrhythmics; antiarthritics; antiasthmatic agents; anticholinergics; anticonvulsants; antidiabetic agents; antidiarrheals; antihelminthics; antihistamines; antiinflammatory agents; antimigraine preparations; antinauseants; antineoplastics; antiparkinsonism drugs; antipruritics; antipsychotics; antipyretics; antisense agents; antispasmodics; cardiovascular preparations including calcium channel blockers and beta-blockers such as pindolol; antihypertensives; central nervous system stimulants; cough and cold preparations, including decongestants; diuretics; gastrointestinal drugs, including H₂-receptor antagonists; sympathomimetics; hormones such as estradiol and other steroids, including corticosteroids; hypnotics; immunosuppressives; muscle relaxants; parasympatholytics; psychostimulants; sedatives; tranquilizers; thrombolytics; neuroprotectants; radical scavengers and vasodilators.

As used herein, the term “restless leg syndrome” encompasses a disorder that typically occurs during sleep or rest, or just before sleep or rest, or periods of inactivity, and which is characterized by uncomfortable sensations in the legs. Examples of uncomfortable sensations in the legs include, but are not limited to, pulling, drawing, crawling, wormy, boring, tingling, pins and needles, prickly and sometimes painful sensations that are usually accompanied by an overwhelming urge to move the legs. As used herein, the term “restless leg syndrome” also encompasses Ekbom Syndrome, Wittmaack-Ecbom Syndrome, Hereditary Acromelalgia, Anxieties Tibialis, periodic limb movements in sleep (PLMS), and periodic limb movement disorder (PLMD).

“Essentially stable plasma tyrosine level” refers to an elevated plasma tyrosine level that is measurable and detectable and which does not increase or decrease by more than about 40%, more than about 30%, more than about 20%, more than about 10% or more than about 5% during the period of its measurement. Fluctuations in plasma tyrosine concentrations of up to 100% of the baseline concentrations may be anticipated from dietary variations, but these are not of significant impact on the stable elevated levels of plasma tyrosine.

The compounds of use in the present invention can be used to inhibit or reduce HPPD activity. In these contexts, inhibition and reduction of HPPD refers to a lower level of the measured activity relative to a control experiment in which the enzyme or cells or animals are not treated with the test compound. In particular embodiments, the inhibition or reduction in the measured activity is at least a 10% reduction or inhibition. One of skill in the art will appreciate that reduction or inhibition of the measured activity of at least 20%, 50%, 75%, 90% or 100% may be preferred for particular applications.

### II. Overview

The present invention discloses compositions and methods for the treatment or prevention of restless leg syndrome (RLS). The compositions of the invention comprise a compound that can inhibit 4-hydroxyphenylpyruvate dioxygenase (HPPD). The methods of the invention comprise administering to a patient in need of such treatment or prevention a therapeutically effective amount of a HPPD inhibitor in order to treat or prevent RLS, or a symptom or cause thereof.

In one aspect of the invention, methods of treating or preventing restless leg syndrome are provided, which comprise administering to a subject in need of such treatment or prevention a therapeutically or prophylactically effective amount of 2-(2-nitro-4-trifluoromethylbenzoyl)-1,3-cyclohexanedione, or a pharmaceutically acceptable salt, solvate, hydrate, clathrate, or prodrug thereof. In another aspect of the invention, the methods comprise administering to a subject in need of such treatment or prevention a therapeutically or prophylactically effective amount of an HPPD inhibitor (e.g., 2-(2-nitro-4-trifluoromethylbenzoyl)-1,3-cyclohexanedione) in combination with at least one compound falling within the classification(s) of anxiolytics, hypnotics or sedatives. In various embodiments, the invention methods include administering a composition including an HPPD inhibitor (e.g., 2-(2-nitro-4-trifluoromethylbenzoyl)-1,3-cyclohexanedione) in combination with benzodiazepines (e.g., clonazepam, carbamazepine), pergolide, carbidopa, levodopa, oxycodone, carbamazepine, gabapentin, magnesium, iron, vitamins, or pharmaceutically acceptable salts, solvates, hydrates, clathrates, prodrugs, optically and pharmacologically active stereoisomers. The invention also provides such compositions.

The invention provides a method of treating or preventing RLS at night—during or prior to sleep—and during the day, e.g., at rest during the day or during periods of inactivity. The includes administering to a subject with RLS a therapeutically effective amount of an HPPD inhibitor.

The invention also provides compositions and methods for ameliorating, treating or preventing sleep disorders and abnormalities and/or improving sleep quantity or adequacy. The methods of the invention include administering to a subject in need to such treatment, prevention or improvement an HPPD inhibitor (e.g., 2-(2-nitro-4-trifluoromethylbenzoyl)-1,3-cyclohexanedione) in a therapeutically effective amount. In various embodiments, the methods do not induce or require sedation. In an exemplary embodiment, the invention provides a method producing improved onset and duration of sleep. In various embodiments, the method of the invention does not induce any CNS effects other those set forth herein.

Exemplary methods of the invention comprise administering to a subject in need of such treatment or prevention of sleep disorder or abnormality or improvement in sleep quantity or adequacy a therapeutically or prophylactically effective amount of an HPPD inhibitor (e.g., 2-(2-nitro-4-trifluoromethylbenzoyl)-1,3-cyclohexanedione) in combination with at least one compound falling within the classification(s) of anxiolytics, hypnotics or sedatives. In various embodiments, the invention methods include administering a composition including an HPPD inhibitor (e.g., 2-(2-nitro-4-trifluoromethylbenzoyl)-1,3-cyclohexanedione) in combination with benzodiazepines (e.g., clonazepam, carbamazepine), pergolide, carbidopa, levodopa, oxycodone, carbamazepine, gabapentin, magnesium, iron, vitamins, or pharmaceutically acceptable salts, solvates, hydrates, clathrates, prodrugs, optically and pharmacologically active stereoisomers.

### III. Compounds

In one aspect of the invention, compositions and methods for their use to inhibit 4-hydroxyphenylpyruvate dioxygenase (HPPD) are provided for the treatment of RLS. The HPPD inhibitor can be any compound that is known to inhibit HPPD.

According to the present invention there is provided the use of 2-(2-nitro-4-trifluoromethylbenzoyl)-1,3-cyclohexanedione (Compound I):

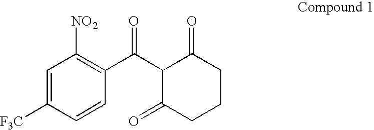

or a pharmaceutically acceptable salt, solvate, hydrate, clathrate, or prodrug thereof, in the manufacture of a medicament for use in the treatment or prevention of RLS, sleep disorder or abnormality, or in the improvement of sleep quantity or adequacy. 2-(2-Nitro-4-trifluoromethylbenzoyl)-1,3-cyclohexanedione can exist in one or more tautomeric forms, which are readily inter-convertible by keto-enol tautomerism. It is to be understood that the invention includes the use of 2-(2-nitro-4-trifluoromethylbenzoyl)-1,3-cyclohexanedione in any of such tautomeric forms or as a mixture thereof.

2-(2-Nitro-4-trifluoromethylbenzoyl)-1,3-cyclohexanedione is acidic and can form pharmaceutically acceptable salts with a wide variety of bases.

Exemplary salts of 2-(2-nitro-4-trifluoromethylbenzoyl)-1,3-cyclohexanedione suitable for use as active ingredients in pharmaceutical compositions according to the invention include, for example, pharmaceutically acceptable base-addition salts, for example, alkali metal (such as potassium or sodium), alkaline earth metal (such as calcium or magnesium) and ammonium salts, and salts with organic bases giving physiologically acceptable cations (such as salts with methylamine, dimethylamine, trirnethylamine, piperidine and morpholine).

2-(2-Nitro-4-trifluoromethylbenzoyl)-1,3-cyclohexanedione can be synthesized by conventional procedures of organic chemistry already known for the production of structurally analogous materials. Typically, 2-nitro-4-trifluororomethylbenzoic acid can be obtained, for example, as described by Haupstein et al. J. Am. Chem. Soc. (1954) 76: 1051, or by one of the general methods well known to the skilled person.

Thus, for example, 2-(2-nitro-4-trifluoromethylbenzoyl)-1,3-cyclohexanedione can be synthesized by reaction of 2-nitro-4-trifluoromethylbenzoyl chloride with cyclohexane-1,3-dione in the presence of acetone cyanohydrin and a suitable base such as triethylamine.

The starting 2-nitro-4-trifluoromethylbenzoyl chloride may itself be obtained from the corresponding benzoic acid, for example by reaction with thionyl chloride or oxalyl chloride as is described in Reagents for Organic Synthesis, (J Wiley and Sons, 1967; editors: Fieser L. F. and Fieser M.; Vol 1, pp. 767-769) and is generally used without special purification.

### IV. Dosage

The amount of HPPD inhibitor, such as, 2-(2-nitro-4-trifluoromethylbenzoyl)-1,3-cyclohexanedione, that can be combined with one or more excipients to produce a single dosage form may vary depending upon the subject and the particular route of administration.

In an exemplary embodiment of the invention, the HPPD inhibitor is given at a dosage that produces a stable plasma tyrosine level. An exemplary dosage is the minimum dosage necessary to achieve a stable plasma tyrosine level. In various embodiments, a subject is dosed with the inhibitor at an induction/loading dosage for a first dosing period. Following the first dosing period, the subject is provided a dosage lower than the induction/loading dosage for a second dosing period. In exemplary embodiments, plasma tyrosine levels do not increase or decrease more than about 40%, more than about 30%, more than about 20, more than about 10% or more than about 5% during the second dosing period. In various embodiments, the inhibitor is administered once every two days, once every three days, once every four days or even less frequently. In an exemplary embodiment, under this intermittent dosing regimen, plasma levels of tyrosine do not increase or decrease more than about 40%, more than about 30%, more than about 20, more than about 10% or more than about 5% between administration of a dosage of HPPD inhibitor.

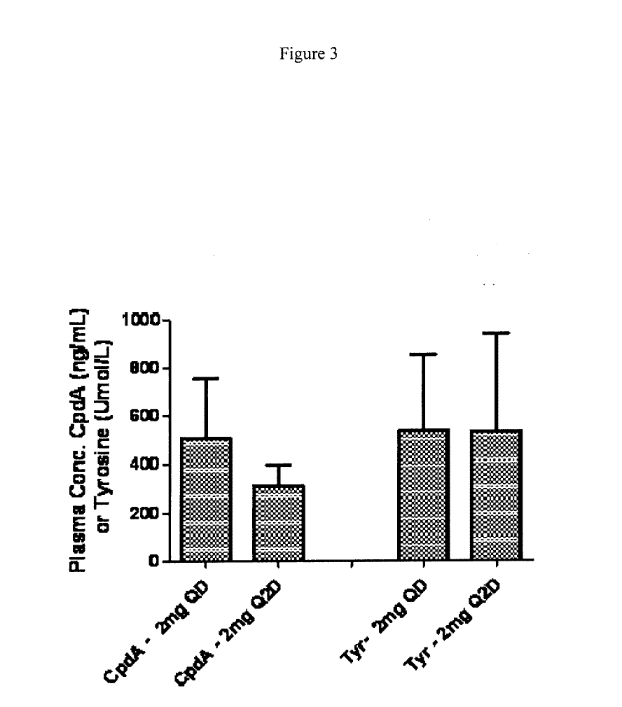

FIG. 3 provides an example of plasma tyrosine levels which are essentially identical and independent of whether the HPPD inhibitor is administered once a day or once every two days. This figure shows steady state plasma tyrosine levels following administration of an exemplary HPPD inhibitor (2-(2-nitro-4-trifluoromethylbenzoyl)-1,3-cyclohexanedione) dosed a once a day at 2 mg and once in 2 days at 2 mg. Though the plasma levels of (2-(2-nitro-4-trifluoromethylbenzoyl)-1,3-cyclohexanedione) are lower in the once in two day regimen, the tyrosine levels in the once a day and once in two days are substantially similar

In an exemplary embodiment, the invention provides a method of achieving an essentially stable plasma level of tyrosine in a subject. The method includes administering to the subject an amount of an HPPD inhibitor at a dosing frequency sufficient to provide the essentially stable plasma level of tyrosine. In an exemplary embodiment, the HPPD inhibitor is administered not more than once every two days and the plasma tyrosine level is essentially stable during the periods between administration.

Generally, a formulation intended for oral administration to humans will generally contain for example from about 0.01 mg to about 100 mg of the active agent (e.g., HPPD inhibitor), preferably about 0.05 mg to about 5 mg per dose of the active agent combined with an appropriate amount of excipient(s). However, exemplary dosage unit forms generally contain about 0.1 mg to about 500 mg, from about 0.3 to about 300 mg, from about 0.5 to about 150 mg, from about 1 to about 100 mg, from about 2 to about 50 mg, from about 3 to about 25 mg and from about 4 to about 10 mg of an active ingredient.

In various embodiments, the invention provides an oral formulation for administration to humans having from about 0.01 mg to about 10 mg of the active agent (e.g., HPPD inhibitor), for example, from about 0.05 mg to about 5 mg, which is optionally combined with an appropriate amount of excipient(s).

In various embodiments, the invention provides an oral unit dose formulation for administration to humans having from about 1 mg to about 2 mg of the active agent (e.g., HPPD inhibitor). This unit dosage form can be a daily dosage form or can be administered less than daily (e.g., once every other day).

More specifically, a formulation comprising 2-(2-nitro-4-trifluoromethylbenzoyl)-1,3-cyclohexanedione, for example, intended for oral administration to humans will generally contain for example from about 0.01 mg to about 500 mg of active agent combined with an appropriate amount of excipient(s), more preferably, about 0.1 mg to about 500 mg, from about 0.3 to about 300 mg, from about 0.5 to about 150 mg, from about 1 to about 100 mg, from about 2 to about 50 mg, from about 3 to about 25 mg and from about 4 to about 10 mg of 2-(2-nitro-4-trifluoromethylbenzoyl)-1,3-cyclohexanedione.

However, it will be readily understood that it may be necessary to vary the dose of the active ingredient administered in accordance with well known medical practice in order to take account of the nature and severity of the condition or disease under treatment, any concurrent therapy, and of the age, weight, genotype and sex of the patient receiving treatment.

Generally, in therapeutic use, it is envisaged that a composition according to the invention would be administered so that a dose of the HPPD inhibitor (or of an equivalent amount of a pharmaceutically acceptable salt thereof) is received which is generally in the range of about 0.01 to about 500 mg/day, about 0.1 to about 100 mg/day, about 0.5 to about 10 mg/day and about 1 to about 5 mg/day of active agent daily given, if necessary in divided doses.

More specifically, for a composition comprising 2-(2-nitro-4-trifluoromethylbenzoyl)-1,3-cyclohexanedione, in therapeutic use, a composition according to the invention is administered so that a dose of the HPPD inhibitor (or of an equivalent amount of a pharmaceutically acceptable salt thereof) is received which is generally in the range 0.01 to 100 mg/day. More specifically, from between 0.05 to 10 mg/day and 0.1 to 5 mg/day or 0.01 to 1 mg of 2-(2-nitro-4-trifluoromethylbenzoyl)-1,3-cyclohexanedione daily given if necessary in divided doses.

More specifically, for a composition comprising 2-(2-nitro-4-trifluoromethylbenzoyl)-1,3-cyclohexanedione, in therapeutic use, it is envisaged that a composition according to the invention could be administered intermittently, on a regimen of alternate days, twice weekly or weekly, so that a dose of the HPPD inhibitor (or of an equivalent amount of a pharmaceutically acceptable salt thereof) is received which is generally in the range 0.01 to 100 mg/week. More specifically, from between 0.05 to 20 mg/week and 0.1 to 5 mg/day of 2-(2-nitro-4-trifluoromethylbenzoyl)-1,3-cyclohexanedione. The regimen of choice would provide the lowest effective concentration of the HPPD inhibitor which attain a sustained elevation of plasma tyrosine in the range of 5 to 10 times above normal ranges, and thus provide a continuous source of tyrosine to produce elevated brain tyrosine concentrations.

In addition to assessment of the overall condition of the patient, the effects of administration of the HPPD inhibitor thereof may be monitored by standard clinical chemical and blood assays, or by monitoring plasma tyrosine as a surrogate of brain tyrosine, liver function tests, and active agent concentrations.

In a still further aspect of the invention there is provided a method of treating and/or preventing RLS comprising administering to the patient a pharmaceutically effective amount of a HPPD inhibitor.

In a particular embodiment of the invention said disease is treated. In a still further embodiment of the invention the patient is a human being. In a still further embodiment of the invention the disease can be RLS, Ekbom Syndrome, Wittmaack-Ecbom Syndrome, Hereditary Acromelalgia, Anxieties Tibialis, periodic limb movements in sleep (PLMS), and periodic limb movement disorder (PLMD). In a still further embodiment of the invention said HPPD inhibitor is as described above. In a particular embodiment said inhibitor comprises 2-(2-nitro-4-trifluoromethylbenzoyl)-1,3-cyclohexanedione or a pharmaceutically acceptable salt, solvate, hydrate, clathrate, or prodrug thereof.

While the compound of the present invention has a superior effect even when used solely, the effect can be further promoted by using the compound in combination with other pharmaceutical preparations and therapies. Examples of the preparation and therapy to be combined include, but not limited to benzodiazipines (e.g., clonazepam, carbamazepine), pergolide, carbidopa, levodopa, oxycodone, gabapentin, magnesium, iron, vitamins, narcotics, dopamine agonists, or pharmaceutically acceptable salts, solvates, hydrates, clathrates, prodrugs, optically and pharmacologically active stereoisomers. Preferably, the combination has a synergistic effect.

As one of skill in the art will recognize, the timing of administering the dosage containing the HPPD inhibitor can vary. In one aspect of the invention, the HPPD inhibitor are administered after the identification of symptoms of RLS. The administration of the HPPD inhibitor can be initiated after the onset of the symptoms. In another aspect of the invention, the HPPD inhibitor is administered to the patient concurrently with another pharmacologically active compound. Preferably, the HPPD inhibitor is administered for about 1 month to about 3 months to facilitate recovery. Preferably, the compounds, and compositions comprising the compounds are administered up to about 12 months or longer, or, even more preferably, administered continuously.

In another aspect of the invention, the HPPD inhibitor are administered prophylactically to patients with a predisposition towards RLS. The administration of the HPPD inhibitor can be initiated before the onset of the symptoms. In another aspect of the invention, the HPPD inhibitor are administered to the patient concurrently with another pharmacologically active compound. Preferably, the HPPD inhibitor is administered for about 1 month to about 3 months to facilitate recovery. Preferably, the compounds, and compositions comprising the compounds are administered up to about 12 months or longer, or, even more preferably, administered continuously.

### V. Pharmaceutical Formulations and Modes of Administration

The methods described herein use pharmaceutical compositions comprising the HPPD inhibitors described above, where the compositions preferably comprise 2-(2-nitro-4-trifluoromethylbenzoyl)-1,3-cyclohexanedione, optionally in combination with another compound with which it has synergistic effect, together with one or more pharmaceutically acceptable excipients or vehicles, and optionally other therapeutic and/or prophylactic ingredients. Such excipients include liquids such as water, saline, glycerol, polyethyleneglycol, hyaluronic acid, ethanol, etc. Suitable excipients for non-liquid formulations are also known to those of skill in the art. Pharmaceutically acceptable salts can be used in the compositions of the present invention and include, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients and salts is available in Remington's Pharmaceutical Sciences, 18th Edition (Easton, Pa.: Mack Publishing Company, 1990).

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, i.e., a pH in the physiologically acceptable range. Examples of buffer solutions include saline, phosphate buffered saline, Tris buffered saline, Hank's buffered saline, and the like.

Depending on the intended mode of administration, the pharmaceutical compositions may be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, may include other pharmaceutical agents, adjuvants, diluents, buffers, etc.

The invention includes a pharmaceutical composition comprising a compound of the present invention including isomers, racemic or non-racemic mixtures of isomers, or pharmaceutically acceptable salts or solvates thereof together with one or more pharmaceutically acceptable carriers, and optionally other therapeutic and/or prophylactic ingredients.

In general, the compounds of this invention will be administered in a therapeutically effective amount by any of the accepted modes of administration. Suitable dosage ranges depend upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compounds of this invention for a given disease.

The pharmaceutical compositions and dosage forms of this invention are particularly useful in the methods herein, and may be suitable for oral, mucosal (e.g., nasal, sublingual, buccal, rectal, and vaginal), parenteral (e.g., intravenous, intramuscular or subcutaneous), or transdermal administration.

Preferred pharmaceutical compositions and dosage forms comprise 2-(2-nitro-4-trifluoromethylbenzoyl)-1,3-cyclohexanedione or another HPPD inhibitor, or a pharmaceutically acceptable salt, solvate, hydrate, clathrate, or prodrug thereof in an amount from about 0.1 mg to about 500 mg, preferably from about 1 mg to about 100 mg, and more preferably from about 2 mg to about 25 mg. Pharmaceutical compositions and dosage forms of the invention typically also comprise one or more pharmaceutically acceptable excipients or diluents.

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g. aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

For example, oral dosage forms such as tablets may contain excipients not suited for use in parenteral dosage forms. Pharmaceutical compositions of the invention that are suitable for oral administration can be presented as discrete dosage forms, such as, but are not limited to, tablets (e.g., chewable tablets), caplets, capsules, and liquids (e.g., flavored syrups). Such dosage forms contain predetermined amounts of active ingredients, and may be prepared by methods of pharmacy well known to those skilled in the art.

Typical oral dosage forms of the invention are prepared by combining 2-(2-nitro-4-trifluoromethylbenzoyl)-1,3-cyclohexanedione in an admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro-crystalline cellulose, diluents, granulating agents, lubricants, binders, fillers, and disintegrating agents.

A tablet can be prepared by compression or molding. Compressed tablets can be prepared by compressing in a suitable machine 2-(2-nitro-4-trifluoromethylbenzoyl)-1,3-cyclohexanedione in a free-flowing form such as powder or granules, optionally mixed with an excipient. Molded tablets can be made by molding in a suitable machine a mixture of the powdered compound moistened with an inert liquid diluent.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof.

Suitable forms of microcrystalline cellulose include, but are not limited to, the materials sold as AVICEL-PH-101™, AVICEL-PH-103™, AVICEL RC-581™, AVICEL-PH-105™ (available from FMC Corporation, American Viscose Division, Avicel Sales, Marcus Hook, Pa.), and mixtures thereof. An specific binder is a mixture of microcrystalline cellulose and sodium carboxymethyl cellulose sold as AVICEL RC-581™. Suitable anhydrous or low moisture excipients or additives include AVICEL-PH-103™ and Starch 1500 LM.

Examples of fillers suitable for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation, and is readily discernible to those of ordinary skill in the art. Typical pharmaceutical compositions comprise from about 0.5 to about 15 weight percent of disintegrant, preferably from about 1 to about 5 weight percent of disintegrant.

Disintegrants that can be used in pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants that can be used in pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200™, manufactured by W. R. Grace Co. of Baltimore, Md.), a coagulated aerosol of synthetic silica (marketed by Degussa Co. of Plano, Tex.), CAB-O-SILT™ (a pyrogenic silicon dioxide product sold by Cabot Co. of Boston, Mass.), and mixtures thereof. If used at all, lubricants are typically used in an amount of less than about 1 weight percent of the pharmaceutical compositions or dosage forms into which they are incorporated.

The magnitude of a prophylactic or therapeutic dose of an active ingredient in the acute or chronic management of a RLS disorder or condition will vary with the severity of the disorder or condition to be treated and the route of administration.

In an exemplary embodiment, the present invention provides a unit dosage formulation having an amount of an HPPD inhibitor which is therapeutically effective to treat or prevent RLS and/or its symptoms; or treat or prevent sleep disorders and/or improve sleep quantity and/or adequacy according an art-recognized measure, e.g., the MOS Sleep Scale. This unit dosage formulation optional includes another pharmacologically active compound, which is efficacious in treating or preventing the disease or condition the HPPD inhibitor is intended to treat or, optionally, is of use to treat a disease or condition which is co-morbid with the disease or condition the HPPD inhibitor is intended to treat.

### Kits

This invention further comprises kits which, when used by the medical practitioner, can simplify the administration of appropriate amounts of active ingredients to a patient.

A typical kit of the invention comprises a unit dosage form of a HPPD inhibitors of the invention, such as 2-(2-nitro-4-trifluoromethylbenzoyl)-1,3-cyclohexanedione, or a pharmaceutically acceptable prodrug, salt, solvate, hydrate, or clathrate thereof, and a unit dosage form of an additional pharmacologically active compound. Examples of additional pharmacologically active compounds are disclosed herein.

Kits of the invention can further comprise devices that are used to administer the active ingredients. Examples of such devices include, but are not limited to, syringes, drip bags, patches, and inhalers.

Kits of the invention can further comprise pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

In summary, the present invention provides:

A method of treating or preventing restless leg syndrome which comprises administering to a patient in need of such treatment or prevention a therapeutically or prophylactically effective amount of a HPPD inhibitor, or a pharmaceutically acceptable salt, solvate, clathrate, or prodrug thereof. The invention includes, for example, treatment of RLS during the day (e.g., at rest or during inactivity) or night (e.g., during or prior to sleep).

A method of treating or preventing sleep disorders which comprises administering to a patient in need of such treatment or prevention a therapeutically or prophylactically effective amount of a HPPD inhibitor, or a pharmaceutically acceptable salt, solvate, clathrate, or prodrug thereof.

A method of improving sleep quantity or sleep adequacy which comprises administering to a patient in need of such treatment or prevention a therapeutically or prophylactically effective amount of a HPPD inhibitor, or a pharmaceutically acceptable salt, solvate, clathrate, or prodrug thereof.

A method according to any preceding paragraph wherein the HPPD inhibitor is 2-(2-nitro-4-trifluoromethylbenzoyl)-1,3-cyclohexanedione.

A method according to any preceding paragraph which further comprises the administration of another pharmaceutically active compound, e.g., pergolide, carbidopa, levodopa, oxycodone, carbamazepine, or gabapentin, or a pharmaceutically acceptable salt, solvate, hydrate, clathrate, prodrug, optically and pharmacologically active stereoisomer, or pharmacologically active metabolite thereof.

A pharmaceutical composition comprising 2-(2-nitro-4-trifluoromethylbenzoyl)-1,3-cyclohexanedione, or a pharmaceutically acceptable salt, solvate, hydrate, clathrate, or prodrug thereof.

A pharmaceutical composition according to any preceding paragraph, wherein the 2-(2-nitro-4-trifluoromethylbenzoyl)-1,3-cyclohexanedione is optically pure.

The pharmaceutical composition according to any preceding paragraph wherein the 2-(2-nitro-4-trifluoromethylbenzoyl)-1,3-cyclohexanedione is in an amount of from about 0.1 mg to about 60 mg.

A pharmaceutical composition of any preceding paragraph, wherein the 2-(2-nitro-4-trifluoromethylbenzoyl)-1,3-cyclohexanedione is in an amount of from about 2 mg to about 30 mg.

A pharmaceutical composition of any preceding paragraph, wherein the 2-(2-nitro-4-trifluoromethylbenzoyl)-1,3-cyclohexanedione is in an amount of from about 5 mg to about 15 mg.

A pharmaceutical composition according to any preceding paragraph, wherein the pharmaceutical composition is adapted for oral, mucosal, rectal, parenteral, transdermal, or subcutaneous administration.

A pharmaceutical composition according to any preceding paragraph for treating or preventing Restless Leg Syndrome, the composition comprising 2-(2-nitro-4-trifluoromethylbenzoyl)-1,3-cyclohexanedione or a pharmacologically acceptable salt thereof and pharmaceutical excipient.

A pharmaceutical composition according to any preceding paragraph, wherein the 2-(2-nitro-4-trifluoromethylbenzoyl)-1,3-cyclohexanedione or the pharmacologically acceptable salt thereof is present at a concentration of about 0.01 mg to about 50.0 mg.

The pharmaceutical composition of any preceding paragraph of use in a method according to any preceding paragraph.

The invention is illustrated by the following Examples; however, it is not limited by the scope of these Examples.

### EXAMPLES

### Example 1

### Study Design

The study multi-centre, randomized, double-blind, parallel-groups, placebo-controlled study with 2 treatment arms, oral HPPD inhibitor (2-(2-nitro-4-trifluoromethylbenzoyl)-1,3-cyclohexanedione) and placebo. Subjects were randomized to either treatment with an HPPD inhibitor or placebo in a 2:1 ratio. The subjects were dosed with 4 mg on day 1, and 2 mg/day on days 2-14. The total duration of treatment with study drug for each subject was 14 consecutive days ending at the day of the final sleep lab assessment (Day 14), with a follow-up examination 29 days after Baseline.

The objective of this exploratory study was to show proof-of-concept of therapy with an exemplary HPPD inhibitor in moderate to severe idiopathic RLS subjects. It was assessed whether a reduction in motor symptoms and subjective severity ratings of the RLS under 2-(2-nitro-4-trifluoromethylbenzoyl)-1,3-cyclohexanedione therapy compared to placebo could be demonstrated.

Efficacy parameters were changes between Baseline and end of treatment in objective measures of Periodic Limb Movements (PLM) and sleep efficiency and in investigator evaluations of severity of RLS symptoms as well as in subject's report on severity of RLS symptoms, sleep disturbances and depressive feelings. Parameters measured included:
Periodic Limb Movement (PLM) Index, defined as the number of PLM events occurring during time-in-bed (PLMI/h TIB)
Periodic Limb Movements during Sleep Index, defined as the number of PLMs per hour of total sleep time (PLMSI/h TST)
PLM arousal index, defined as the number of PLM with arousals per hour of total sleep time (PLMSAI/h TST)
Periodic Limb Movements during wakefulness index, defined as the number of PLMs per hour occurring during wakefulness within time-in-bed (PLMWI/h TIB)
Sleep Efficiency (SE) expressed as the percentage total sleep time (TST) of total time in bed (TIB)
Total Sleep Time (TST)
Total number of arousals per TST
Sleep latency
Restless Legs Scale—6 items (RLS-6) (Kohnen et al, 2004)
IRLS total score change from start of treatment to end
Time to requiring rescue medication (defined as time to dropout due to the need of rescue medication for intolerable RLS symptoms)
Physicians' Global Impression, rated by the Clinical Global Impressions Scale (CGI): severity, change and treatment effect
CGI responders defined as the proportion of subjects who improved “much” or “very much” on CGI item “change”.
Patients' Global Impression (PGI) scale Item 1 (efficacy)
Medical Outcomes Study (MOS) Sleep Scale
Beck Depression Inventory (BDI)
In addition, a patient diary was to be completed between days 8 and 15
HPPD inhibitor plasma concentrations at Baseline, end of treatment and follow-up
Tyrosine plasma concentration at Baseline, end of treatment and follow-up.

### Results

Physicians' and patients' ratings of RLS symptom severity, sleep impairment and mood as well as the investigators' and patients' global evaluation of changes from Baseline favored 2-(2-nitro-4-trifluoromethylbenzoyl)-1,3-cyclohexanedione over placebo. As observed from the log-transformed values on PSG measurements the PLM index values yielded a slightly larger improvement under an exemplary HPPD inhibitor, 2-(2-nitro-4-trifluoromethylbenzoyl)-1,3-cyclohexanedione, as compared to placebo.

Results from the IRLS scale showed that more subjects (M-ITT) under 2-(2-nitro-4-trifluoromethylbenzoyl)-1,3-cyclohexanedione reported only mild or moderate RLS symptoms at EoT as compared to placebo (9 [64.3%] vs. 2 [28.6%] subjects). Analysis of the IRLS total score and IRLS severity score (but not of the IRLS impact score) showed numerical differences between Baseline and EoT favouring 2-(2-nitro-4-trifluoromethylbenzoyl)-1,3-cyclohexanedione treatment, especially the results from severity score were noteworthy: difference 2-(2-nitro-4-trifluoromethylbenzoyl)-1,3-cyclohexanedione-placebo [M-ITT]: LS mean: −3.46; 95%-CI: −9.75; 2.84; p=0.2636.

The median (mean) differences between Baseline and EoT visit in all subscales of the RLS-6 indicated that subjects treated with 2-(2-nitro-4-trifluoromethylbenzoyl)-1,3-cyclohexanedione experienced more benefit than those treated with placebo. Most notably, RLS symptoms when falling asleep (difference 2-(2-nitro-4-trifluoromethylbenzoyl)-1,3-cyclohexanedione-placebo [M-ITT]: LS mean: −2.62; 95%-CI: −5.24; −0.01; p=0.0494 [two-sided from ANCOVA]) or during the night (difference 2-(2-nitro-4-trifluoromethylbenzoyl)-1,3-cyclohexanedione-placebo [M-ITT]: LS mean: −1.77; 95%-CI: −4.52; 0.97; p=0.1910 [two-sided from ANCOVA]) were decreased.

Results favoring 2-(2-nitro-4-trifluoromethylbenzoyl)-1,3-cyclohexanedione over placebo were found in several MOS subscales. See, Example 2, hereinbelow.

Mean differences between Baseline and EoT visit showed larger reduction of depressive symptoms in the 2-(2-nitro-4-trifluoromethylbenzoyl)-1,3-cyclohexanedione group as compared to placebo.

Results from the CGI severity item indicated larger improvement on 2-(2-nitro-4-trifluoromethylbenzoyl)-1,3-cyclohexanedione than on placebo treatment. In both groups, there were subjects at EoT with moderate to severe illness (7 [50.0%] vs. 5 [71.4%] subjects), but only in the 2-(2-nitro-4-trifluoromethylbenzoyl)-1,3-cyclohexanedione group 3 (21.4%) subjects were not at all or only borderline ill (M-ITT). With respect to change of condition, on 2-(2-nitro-4-trifluoromethylbenzoyl)-1,3-cyclohexanedione 7 (50.0%) subjects had very much or much improved (responder analysis), but only 2 (28.6%) placebo subjects (M-ITT). Treatment effect was rated as very good or moderate in 8 (57.1%) 2-(2-nitro-4-trifluoromethylbenzoyl)-1,3-cyclohexanedione and 3 (42.9%) placebo treated subjects. The results from PGI indicated that more subjects of the 2-(2-nitro-4-trifluoromethylbenzoyl)-1,3-cyclohexanedione group as compared to the placebo group (7 [50.0%] vs. 2 [28.6%]) assessed their therapy as efficacious.

The trends observed in the RLS-6 scales were paralleled by an even stronger signal in the patient diary data. Treatment effects were especially observed for improvement of RLS symptoms during the day (difference 2-(2-nitro-4-trifluoromethylbenzoyl)-1,3-cyclohexanedione-placebo [M-ITT]: LS mean: −2.57; 95%-CI: 4.87; −0.27; p=0.0304 [two-sided from ANCOVA]), when falling asleep (difference 2-(2-nitro-4-trifluoromethylbenzoyl)-1,3-cyclohexanedione-placebo [M-ITT]: LS mean: −2.64; 95%-CI: −5.15; −0.13; p=0.0402 [two-sided from ANCOVA]) and during the day when active (difference 2-(2-nitro-4-trifluoromethylbenzoyl)-1,3-cyclohexanedione-placebo [M-ITT]: LS mean: −1.28; 95%-CI: −2.52; −0.04; p=0.0437 [two-sided from ANCOVA]). Trends were found for the improvement of RLS symptoms during the night and during the day at rest, or during inactivity.

### Example 2

### Improvement in Sleep Parameters

Subjects with RLS received either placebo (7 subjects) or an HPPD inhibitor (13 subjects) given as 4 mg on day 1, then 2 mg per day for days 2-14 of the trial. Various parameters relevant to RLS were monitored during the course of treatment, in particular the quality of their sleep patterns using a standard assessment scale (Medical Outcomes Study (MOS) Sleep Scale). Individuals were asked to complete a standard evaluation of their ‘normal’ sleep experience at the start of the trial (Baseline) and again at the end of treatment (EoT), with the various parameters scored according to the attached document. Trends to positive effects were seen in all the parameters measured, reaching statistical significance for the “Sleep Adequacy” parameter.

### MOS Sleep Scale

The MOS Sleep Scale is a comprehensive subject completed sleep questionnaire consisting of 12 items (Hays and Stewart, 1992; Hays et al., 2005). Each item measures a unique aspect or characteristic of sleep. Overall, the MOS Sleep Scale measures the following:

1. Time to fall asleep

2. Total sleep time (in hours)

3. Sleep restlessness

4. Enough sleep, feel rested

5. Awakening during sleep short of breath or with headache

6. Feeling drowsy or sleepy during the day

7. Trouble falling asleep

8. Awakening during sleep time with trouble falling asleep again

9. Trouble staying awake

10. Snoring during sleep

11. Taking naps

12. Amount of sleep needed

All items, except for item 2, were transformed in a ‘0-100’ scale.

For the analysis of the MOS sleep scale, the following subscales were determined as means of the involved items (in part, after reversing the ratings in the ‘0-100’ scale, denoted by (R) in the item sequence):

Sleep disturbance (items 1, 3(R), 7(R), 8(R))

Snoring (10(R))

Sleep short of breath or headache (5(R))

Sleep adequacy (4(R), 12(R))

Sleep somnolence (6(R), 9(R), 11(R))

Sleep problems index I (4, 5(R), 7(R), 8(R), 9(R), 12)

Sleep problems index II (1, 3(R), 4, 5(R), 6(R), 7(R), 8(R), 9(R), 12)

Additionally sleep quality was assessed as duration of sleep:

Sleep quantity (item 2, raw scores);

and optimality of sleep, whereas optimal sleep was defined as sleep of 7-8 hours:

Optimal sleep (item 2, ‘yes’ (7-8 hours)/‘no’ (else)).

These sub-scores were to be analyzed as efficacy variables in this trial.

Subjects should answer each item in reference to the week prior to their visit. If the timeframe since the last visit is <1 week (e.g., in the event of premature discontinuation), the subject should answer each subscale in reference to the days since their last visit.

Subjects self-reported the length of time it took for them to fall asleep and the average number of hours of sleep each night. Scores for all other items range from 1 (all of the time) to 6 (none of the time).

Each item was scored by the subject. Investigators or clinic personnel could assist the subject if he/she had difficulty understanding the items or locating the appropriate score.

The MOS Sleep Scale was completed at Baseline (Visit 2) and final examination (Visit 3).

### MOS Sleep Scale

Baseline values of the MOS sleep scales were comparable for both treatment groups. Absolute values at Baseline and EoT as well as changes between Baseline and EoT visit (Visit 3) in all MOS subscales are displayed in 1 for PPS.

TABLE 1
Results of the MOS Sleep scale (PPS) - change from Baseline to EoT

| | | | HPPD Inhibitor | | Placebo | |
|---|---|---|---|---|---|---|
| | | | n = 13 | | n = 7 | |
| | | Statistics | absolute | change | absolute | change |
| Sleep Disturbance | BL | M ± SD | 76.8 ± 14.3 | | 78.0 ± 20.4 | |
| Scale | | Median | 78.8 | | 76.3 | |
| | | Range | 57.5-95.0 | | 51.3-100.0 | |
| | EoT | M ± SD | 41.8 ± 16.7 | −35.0 ± 27.5 | 59.1 ± 22.5 | −18.9 ± 24.5 |
| | | Median | 43.8 | −46.3 | 60.0 | −16.3 |
| | | Range | 10.0-72.5 | −68.8-11.3 | 25.0-81.3 | −70.0-5.0 |
| Snoring Scale | BL | M ± SD | 26.2 ± 20.6 | | 31.4 ± 28.0 | |
| | | Median | 20.0 | | 40.0 | |
| | | Range | 0.0-80.0 | | 0.0-80.0 | |
| | EoT | M ± SD | 20.0 ± 16.3 | −6.2 ± 15.0 | 40.0 ± 38.3 | 8.6 ± 41.4 |
| | | Median | 20.0 | 0.0 | 40.0 | 0.0 |
| | | Range | 0.0-40.0 | −40.0-20.0 | 0.0-100.0 | −20.0-100.0 |
| Short of | BL | M ± SD | 16.9 ± 18.0 | | 22.9 ± 29.3 | |
| Breath Scale | | Median | 20.0 | | 20.0 | |
| | | Range | 0.0-40.0 | | 0.0-80.0 | |
| | EoT | M ± SD | 9.2 ± 15.5 | −7.7 ± 19.2 | 17.1 ± 24.3 | −5.7 ± 9.8 |
| | | Median | 0.0 | 0.0 | 0.0 | 0.0 |
| | | Range | 0.0-40.0 | −40.0-20.0 | 0.0-60.0 | −20.0-0.0 |
| Sleep Adequacy | BL | M ± SD | 20.0 ± 26.1 | | 10.0 ± 10.0 | |
| Scale | | Median | 10.0 | | 10.0 | |
| | | Range | 0.0-100.0 | | 0.0-20.0 | |
| | EoT | M ± SD | 46.9 ± 25.9 | 26.9 ± 35.5 | 18.6 ± 24.1 | 8.6 ± 28.0 |
| | | Median | 50.0 | 30.0 | 10.0 | 0.0 |
| | | Range | 0.0-80.0 | −50.0-80.0 | 0.0-70.0 | −10.0-70.0 |
| Somnolence | BL | M ± SD | 34.9 ± 20.8 | | 54.3 ± 16.1 | |
| Scale | | Median | 33.3 | | 60.0 | |
| | | Range | 0.0-73.3 | | 26.7-73.3 | |
| | EoT | M ± SD | 24.6 ± 19.7 | −10.3 ± 16.0 | 42.9 ± 31.0 | −11.4 ± 23.3 |
| | | Median | 13.3 | −13.3 | 53.3 | −6.7 |
| | | Range | 6.7-66.7 | −33.3-13.3 | 0.0-80.0 | −53.3-20.0 |
| Sleep Problems | BL | M ± SD | 60.5 ± 13.0 | | 70.0 ± 11.4 | |
| Index I | | Median | 60.0 | | 73.3 | |
| | | Range | 30.0-76.7 | | 56.7-86.7 | |
| | EoT | M ± SD | 36.7 ± 16.3 | −23.8 ± 20.3 | 59.0 ± 20.2 | −11.0 ± 19.5 |
| | | Median | 33.3 | −20.0 | 63.3 | 0.0 |
| | | Range | 10.0-70.0 | −60.0-3.3 | 23.3-86.7 | −53.3-0.0 |
| Sleep Problems | BL | M ± SD | 63.4 ± 11.7 | | 73.1 ± 14.3 | |
| Index II | | Median | 62.2 | | 75.0 | |
| | | Range | 36.7-80.0 | | 53.9-91.1 | |
| | EoT | M ± SD | 37.9 ± 16.8 | −25.5 ± 19.9 | 57.7 ± 20.6 | −15.4 ± 22.0 |
| | | Median | 32.8 | −23.9 | 62.2 | −9.4 |
| | | Range | 11.1-74.4 | −61.7-−1.7 | 20.0-82.8 | −64.4-0.0 |
| Sleep Quantity | BL | M ± SD | 4.3 ± 1.3 | | 3.3 ± 1.5 | |
| | | Median | 4.0 | | 3.0 | |
| | | Range | 3.0-7.0 | | 1.0-5.0 | |
| | EoT | M ± SD | 6.1 ± 1.0 | 1.8 ± 1.1 | 4.6 ± 1.9 | 1.3 ± 1.4 |
| | | Median | 6.0 | 2.0 | 5.5 | 1.0 |
| | | Range | 4.0-8.0 | 0.0-3.0 | 2.0-6.5 | −1.0-3.0 |

Remarks:
M: arithmetic mean,
SD: standard deviation, except for the subscales Sleep quantity and Sleep adequacy, higher values indicate stronger sleep problems

Favorable results for use of an HPPD inhibitor could be found in several MOS subscales, especially for sleep adequacy, which has proven to be very sensitive for RLS symptomatology in a former RLS trial on sleep improvement by ropinirole treatment (Allen et al., 2004) (difference HPPD inhibitor—placebo [M-ITT]: LS mean: 26.97; 95%-CI: 1.30; 52.64; p=0.0405 [two-sided from ANCOVA], d=0.50) but also for reduced sleep disturbance and both sleep problem indices.

For the latter three scales statistical trends favouring treatment with HPPD inhibitor over placebo were found (sleep disturbance: difference HPPD inhibitor—placebo [M-ITT]: LS mean: −17.30; 95%-CI: −35.53; 0.93; p=0.0616 [two-sided from ANCOVA]; d=0.55; sleep problems index I: difference HPPD inhibitor—placebo [M-ITT]: LS mean: −18.75; 95%-CI: −37.80; 0.31; p=0.0534 [two-sided from ANCOVA], d=0.54; sleep problems index II: difference HPPD inhibitor—placebo [M-ITT]: LS mean: −16.78; 95%-CI: −36.18; 2.61; p=0.0857 [two-sided from ANCOVA], d=0.41).

In addition, it was evaluated with the MOS Sleep Scale whether the subjects reported to receive the amount of sleep he/she needed (optimal sleep). At Baseline, only one patient randomized to 2-(2-nitro-4-trifluoromethylbenzoyl)-1,3-cyclohexanedione reported to sleep optimally. At the EoT Visit, 5 (35.7%) patients of the M-ITT (4 of the PPS) treated with 2-(2-nitro-4-trifluoromethylbenzoyl)-1,3-cyclohexanedione reported optimal sleep while none of the placebo patients reported optimal sleep duration (see appendix 16.4, listing 7.4.2).

While the invention has been particularly shown and described with reference to a preferred embodiment and various alternate embodiments, it will be understood by persons skilled in the relevant art that various changes in form and details can be made therein without departing from the spirit and scope of the invention. All printed patents and publications referred to in this application are hereby incorporated herein in their entirety by this reference.

What is claimed is:

1. A method of treating or preventing restless leg syndrome which comprises administering to a patient in need of such treatment or prevention a therapeutically or prophylactically effective amount of a HPPD inhibitor, or a pharmaceutically acceptable salt, solvate, clathrate, or prodrug thereof.

2. The method of claim 1, wherein the HPPD inhibitor is 2-(2-nitro-4-trifluoromethylbenzoyl)-1,3-cyclohexanedione.

3. The method of claim 2, which further comprises the administration of pergolide, carbidopa, levodopa, oxycodone, carbamazepine, or gabapentin, or a pharmaceutically acceptable salt, solvate, hydrate, clathrate, prodrug, optically and pharmacologically active stereoisomer, or pharmacologically active metabolite thereof.

4. A pharmaceutical composition comprising 2-(2-nitro-4-trifluoromethylbenzoyl)-1,3-cyclohexanedione, or a pharmaceutically acceptable salt, solvate, hydrate, clathrate, or prodrug thereof.

5. The pharmaceutical composition of claim 4, wherein the 2-(2-nitro-4-trifluoromethylbenzoyl)-1,3-cyclohexanedione is optically pure.

6. The pharmaceutical composition of claim 4, wherein the 2-(2-nitro-4-trifluoromethylbenzoyl)-1,3-cyclohexanedione is in an amount of from about 0.1 mg to about 60 mg.

7. The pharmaceutical composition of claim 6, wherein the 2-(2-nitro-4-trifluoromethylbenzoyl)-1,3-cyclohexanedione is in an amount of from about 2 mg to about 30 mg.

8. The pharmaceutical composition of claim 7, wherein the 2-(2-nitro-4-trifluoromethylbenzoyl)-1,3-cyclohexanedione is in an amount of from about 5 mg to about 15 mg.

9. The pharmaceutical composition of claim 4, wherein the pharmaceutical composition is adapted for oral, mucosal, rectal, parenteral, transdermal, or subcutaneous administration.

10. A pharmaceutical composition for treating or preventing Restless Leg Syndrome, the composition comprising 2-(2-nitro-4-trifluoromethylbenzoyl)-1,3-cyclohexanedione or a pharmacologically acceptable salt thereof and pharmaceutical excipient.

11. A pharmaceutical composition according to claim 10, wherein the 2-(2-nitro-4-trifluoromethylbenzoyl)-1,3-cyclohexanedione or the pharmacologically acceptable salt thereof is present at a concentration of about 0.01 mg to about 50.0 mg.

12. A method of treating or preventing sleep disorders which comprises administering to a patient in need of such treatment or prevention a therapeutically or prophylactically effective amount of a HPPD inhibitor, or a pharmaceutically acceptable salt, solvate, clathrate, or prodrug thereof.

13. A method according to claim 12 wherein said HPPD inhibitor is 2-(2-nitro-4-trifluoromethylbenzoyl)-1,3-cyclohexanedione or the pharmacologically acceptable salt thereof.

14. A method of improving sleep quantity or sleep adequacy which comprises administering to a patient in need of such treatment or prevention a therapeutically or prophylactically effective amount of a HPPD inhibitor, or a pharmaceutically acceptable salt, solvate, clathrate, or prodrug thereof.

15. A method according to claim 14 wherein said HPPD inhibitor is 2-(2-nitro-4-trifluoromethylbenzoyl)-1,3-cyclohexanedione or the pharmacologically acceptable salt thereof.
